(19) United States
(12) Patent Application Publication
(10) Pub. No.: US 2010/0006453 A1
(43) Pub. Date: Jan. 14, 2010

(54) Detection System

(75) Inventors: Martin Alan Lee, Wiltshisre (GB); Dario Lyall Leslie, Wiltshire (GB)

Correspondence Address: JOHN S. PRATT, ESQ;KILPATRICK STOCKTON, LLP, 1100 PEACHTREE STREET, SUITE 2800, ATLANTA, GA, 30309, US

(73) Assignee: The Secretary of State for Defence, Wiltshire (GB)

(21) Appl. No.: 12/544,036

(22) Filed: Aug. 19, 2009

Related U.S. Application Data
(62) Division of application No. 10/148,711, filed on May 31, 2002.

(30) Foreign Application Priority Data
Dec. 1, 1999 (GB) 9928232.9

(51) Int. Cl. C12Q 1/68; G01N 27/26

(52) U.S. Cl. 205/780.5; 204/403.01

(57) ABSTRACT

A method for detecting a target nucleic acid sequence in a sample, by subjecting it to an amplification [symbol] and taking continuous electrochemical measurements on it during the reaction. The method can be used to determine whet [symbol] amplification reaction has taken place, to quantitate the amount of target in the sample or to determine sequence characteristics. [symbol] disclosed is apparatus for use in the method, comprising (i) an amplification reaction vessel which comprises an electrochemic [symbol] (ii) means for taking continuous electrochemical measurements on a sample contained in the vessel and (iii) tempertature coat [symbol] measurement means, wherein the electrochemical cell comprises an element formed from an electrically conducting plastics [symbol] such as a polymer loaded with an electrically conducting material. Further disclosed is a reaction vessel for use in the appar [symbol] probe for use in the method and a kit for effecting the method.

The present invention provides methods and apparatus for detecting a target polynucleotide in a sample by monitoring an amplification reaction, preferably in a quantitative manner, as well as probes and kits for use in such methods. The methods can also be suitable for the detection of sequence characteristics such as polymorphisms or allelic variation and so may be used in diagnostic methods.

Known fluorescence polymerase chain reaction (PCR) monitoring techniques include both strand specific and generic DNA intercalator techniques that can be used on a few second-generation PCR thermal cycling devices.

Generic methods utilise DNA intercalating dyes that exhibit increased fluorescence when bound to double stranded DNA species. Fluorescence increase due to a rise in the bulk concentration of DNA during amplifications can be used to measure reaction progress and to determine the target molecule copy number. Furthermore, by monitoring fluorescence with a controlled change of temperature, DNA melting curves can be generated, for example, at the end of PCR thermal cycling.

Generic DNA methods monitor the rise in bulk concentration of nucleic acids without any time penalty. A single fluorescent reading can be taken at the same point in every reaction. End point melting curve analysis can be used to discriminate artefacts from amplicon, and to discriminate amplicons. Peaks of products can be seen at concentrations that cannot be visualised by agarose gel electrophoresis.

In order to obtain high resolution melting data, the melt experiment must be performed slowly on existing hardware taking up to five minutes. However, by continually monitoring fluorescence amplification, a 3D image of the hysteresis of melting and hybridisation can be produced. This 3D image is amplicon dependent and may provide enough information for product discrimination.

It has been found that DNA melting curve analysis in general is a powerful tool in optimising PCR thermal cycling. By determining the melting temperatures of the amplicons, it is possible to lower the denaturing temperatures in later PCR cycles to this temperature. Optimization for amplification from first generation reaction products rather than the genomic DNA, reduces artefact formation occurring in later cycles. Melting temperatures of primer oligonucleotides and their complements can be used to determine their annealing temperatures, reducing the need for empirical optimisation.

The generic intercalator methods however are only quasi-strand-specific and are therefore not very useful where strand specific detection is required.

Strand specific methods utilise additional nucleic acid reaction components to monitor the progress of amplification reactions. These methods may use fluorescence energy transfer (PET) as the basis of detection. One or more nucleic acid probes are labelled with fluorescent molecules, one of which is able to act as an energy donor and the other of which is an energy acceptor molecule. These are sometimes known as a reporter molecule and a quencher molecule respectively. The donor molecule is excited with a specific wavelength of light for which it will normally exhibit a fluorescence emission wavelength. The acceptor molecule is also excited at this wavelength such that it can accept the emission energy of the donor molecule by a variety of distance-dependent energy transfer mechanisms. A specific example of fluorescence energy transfer which can occur is Fluorescence Resonance Energy Transfer or “FRET”. Generally the acceptor molecule accepts the mission energy of the donor molecule when they are in close proximity (e.g. on the same, or a neighbouring molecule). The basis of FET or FRET detection is to monitor the changes at donor and acceptor emission wavelengths.

There are two commonly used types of FET or FRET probes, those using hydrolysis of nucleic acid probes to separate donor from acceptor, and those using hybridization to alter the spatial relationship of donor and acceptor molecules.

Hydrolysis probes are commercially available as TaqMan™ probes. These consist of DNA oligonucleotides which are labelled with donor and acceptor molecules. The probes are designed to bind to a specific region on one strand of a PCR product. Following annealing of the PCR primer to this strand, Tag enzyme extends the DNA with 5′ to 3′ polymerase activity. Tag enzyme also exhibits 5′ to 3′ exonuclease activity. TaqMan™ probes are protected at the 3′ end by phosphorylation to prevent them from priming Tag extension. If the TaqMan™ probe is hybridised to the product strand than an extending Tag molecule may also hydrolyse the probe, liberating the donor from acceptor as the basis of detection. The signal in this instance is cumulative, the concentration of free donor and acceptor molecules increasing with each cycle of the amplification reaction.

Hybridisation probes are available in a number of guises. Molecular beacons are oligonucleotides that have complementary 5′ and 3′ sequences such that they form hairpin loops. Terminal fluorescent labels axe in close proximity for FRET to occur when the hairpin structure is formed. Following hybridisation of molecular beacons to a complementary sequence the fluorescent labels are separated, so FRET does not occur, and this forms the basis of detection.

Pairs of labelled oligonucleotides may also be used. These hybridise in close proximity on a PCR product strand bringing donor and acceptor molecules together so that FRET can occur. Enhanced FRET is the basis of detection. Variants of this type include using a labelled amplification primer with a single adjacent probe.

Rapid PCR methods are becoming more prominent with the development of rapid hot air thermal cyclers such as the RapidCycler™ and LightCycler™ from Idaho Technologies Inc. Other rapid PCR devices are described for example in co-pending British Patent Application Nos. 9625442.0 and 9716052.7. The merits of rapid cycling over conventional thermal cycling have been reported elsewhere. Such techniques are particularly useful for example in detection systems for biological warfare where speed of result is important to avoid loss of life or serious injury.

Vessels which may be used in rapid PCR vessels are described in WO-98/24548. In these vessels, heating is provided by means of an electrically conducting polymer which may be integral with the vessel containing the reagents. Such polymers are generally not transparent in nature, which makes the detection of fluorescent signals more difficult. Optical readers including fibre optic wires arranged in the vicinity of the reagents may be required.

Electrochemistry boasts a vast arsenal of electroanalytical techniques including potentiometric, conductometric or amperometric, coulometric, and/or voltametric or polarographic methods. In the past, in the context of analysis of nucleic acids, much work has been reported in the field of voltammetric and polarographic analysis (see for example E. Palecek. Studia Biophysica, 114, (1986) 1-3, p 39-48. J. Hall et al., Biochem, and Mol. Biol. International, 32. 1 (1994), 21-28, Nurnberg et al., Bioelectrical behaviour and deconformation of native DNA at charged interfaces in “Ions in Macromolecules and Biological Systems (Ed. Everett D. H.) (1978) Proc. 29^{th} Colston Symp. Scienechnica, Bristol, K. Hashimoto et al., Anal. Chem. (1994), 66, 3830-3833).

The applicants have fond that these methods can be advantageously used in assays for detecting the presence of particular nucleic acid sequences in a sample.

Thus, a first aspect of the present invention provides a method for detecting a target nucleic acid sequence in a sample, the method comprising subjecting the sample to an amplification reaction, and taking continuous electrochemical measurements on the sample during the amplification reaction.

“Detecting” may be qualitative and/or quantitative, ie, it may involve assessing the presence and/or quantity of the target sequence in the sample.

The target sequence may be, inter alia, an internal control sequence.

In contrast to known electrochemical detection methods, that of the present invention allows measurements to be taken in situ and continuously whilst an amplification reaction progresses, without needing to disturb the sample.

Using a method of this type, there is no need to use complex optical arrangements for detection of products. The reaction can be detected simply by introducing suitable electrochemical elements into the reaction.

The electrochemical measurements may for instance include potentiometric, conductometric or amperometric, coulometric, and/or voltametric or polarographic measurements. For example, the reduction of adenine and cytosine residues at the negatively charged electrode of an electrochemical cell and the oxidation of guanine and adenine residues at the positively charged electrode are electrochemical effects of DNA that could be used to detect amplification products.

The measurements may be used to determine whether and/or to what extent an amplification reaction has taken place, and/or to quantitate the amount of the target nucleic acid sequence in the sample.

They are preferably temperature dependent electrochemical measurements, such as for nucleic acid interactions which are dependent upon the specific temperatures for duplex stabilisation or destabilisation in a particular reaction system. They may thus allow characterization of one or preferably more than one amplification species.

The electrochemical measurements are taken continuously during temperature transitions. This allows quasi-strand specific characterisation of amplification species. This may be achieved by taking continuous measurements during amplification or by end-point measurement.

As the amount of amplicon increases during the reaction, the electrochemical measurement will vary accordingly. “Continuous” measurement includes taking electrochemical measurements at two or more, preferably three or more, discrete time points throughout the reaction, provided they are taken in situ as the amplification reaction proceeds. For example, measurements may be taken at the specific temperatures at which duplexes are known to stabilise or destabilize, so that the accumulation of amplicon can be monitored.

Such measurements can also be used to monitor the kinetics of amplification. These real-time measurements may allow quantification of the amount of target nucleic acid present in the sample as is known in the art.

The amplification reaction used in the method of the invention may be any of the known methods including polymerase chain reaction (PCR), nucleic acid specific based amplification (NASBA), ligase chain reaction (LCR) or a strand displacement amplification (SDA). In particular, the reaction is a PCR.

The amplification is suitably carried out using a pair of primers which are designed such that only the target nucleotide sequence within a DNA strand is amplified as is well understood in the art. The nucleic acid polymerase is suitably a thermostable polymerase such as Taq polymerase.

Suitable conditions under which the amplification reaction can be carried out are well known in the art. The optimum conditions may be variable in each case depending upon the particular amplicon involved, the nature of the primers used and the enzymes employed. They may however be determined in each case by the skilled person. Typical denaturation temperatures are of the order of 95° C., typical annealing temperatures are of the order of 55° C. and extension temperatures are of the order of 72° C.

A second aspect of the present invention provides apparatus in which a method according to the first aspect may be carried out. This apparatus comprises (i) an amplification reaction vessel which comprises an electrochemical cell, (ii) means for taking continuous electrochemical measurements on a sample contained in the amplification reaction vessel and (iii) temperature control and measurement means, wherein the electrochemical cell comprises at least one element formed from an electrically conducting plastics material. The temperature control means preferably allows thermal cycling of the contents of the amplification reaction vessel.

The means for taking electrochemical measurements ideally allows the measurement of an electrochemical parameter, preferably a temperature dependent parameter, as described above.

Electrically conducting polymers, for use as part of the electrochemical cell, are known in the art and may be obtained for example from Caliente Systems Inc. of Newark, USA. Other examples of such polymers are disclosed for instance in U.S. Pat. Nos. 5,106,540 and 5,106,538.

The electrically conducting plastics material may in particular be a polymer loaded with an electrically conducting material. Such conductor-loaded materials are already known and widely available, for instance from the French company RTP, but their electrical conducting properties have not previously been utilised in electrochemical investigations into nucleotide amplification reactions.

A polymer, typically a thermosetting polymer resin such as a polyethylene, polypropylene, polycarbonate or nylon polymer, may contain embedded in it elements of an electrically conducting material such as carbon (usually in the form of fibres) or a metal (copper, for example). These elements may constitute between say 1 and 50% w/w or higher of the electrically conducting plastics material.

Such polymers may be injection moulded and may therefore be used directly to form reaction vessels and their parts. Thus, in apparatus according to the invention, the at least one plastics material element preferably forms part of or in integral with the amplification reaction vessel. More preferably, the amplification reaction vessel is formed from the electrically conducting plastics material, for instance by injection moulding or extrusion. Such vessels are described in WO-98/24548 although not in connection with electrochemical measurements.

Alternatively (and also as described in WO-98/24548), an internal surface of the reaction vessel may be coated with the plastics material, for example by a lamination and/or deposition technique. The plastics material may suitably be provided in the form of a sheet material or film, for example of from 0.01 to 10 mm, preferably from 0.1 to 0.3 mm thick.

The plastics material element is suitably provided with connection points for connection to an electrical supply. Alternatively, an electric current may be induced in the plastics material for example by exposing it, in use, to suitable electrical or magnetic fields.

An electrically conducting plastics material such as those described above often emits heat when an electric current is passed through it. Such a property is preferred in apparatus according to the invention. Since the electrically conducting plastics element may then also function as the temperature control means, again ideally allowing thermal cycling of the contents of the amplification reaction vessel.

The use of electrically conducting plastics materials in accordance with the invention allows a large number of reaction vessels to be processed simultaneously, since each vessel may be separately connected to an electrical source to allow for independent control of the current passing through it. At the same time, the incorporation of electrochemical cell components, and temperature control means, into the fabric of the vessel itself allows relatively simple and compact vessel designs to be achieved.

According to the invention, the amplification reaction may be effected in electrical contact with a working electrode and a secondary electrode. These are suitably provided in a reaction vessel in which the amplification reaction is effected, for instance in apparatus according to the second aspect of the invention. The working and/or the secondary electrode may comprise an electrically conducting plastics material as described above, which may form part or the whole of, or be integral with, the reaction vessel.

The amplification reaction may also be conducted in the presence of a reference electrode. Examples of suitable reference electrodes are metal electrodes in contact with solutions of their own salts such as silver/silver chloride electrodes or a saturated calomel electrode (mercury/mercury chloride in a potassium chloride electrolyte) or others known in the art.

Means for measuring the electrochemical signals from the amplification reaction, such as one or more of a voltammeter, voltmeter or galvanometer, are suitably included in apparatus according to the invention. These devices will be connected into the circuit in a manner appropriate to obtain the desired electrochemical measurements. Examples of such arrangements are illustrated hereinafter, but others would be apparent to the skilled person.

A third aspect of the present invention provides an amplification reaction vessel for use as part of apparatus according to the second aspect or in a method according to the first. The vessel comprises an electrochemical cell which itself comprises at least one element formed from an electrically conducting plastics material.

As described above, the electrically conducting plastics material is preferably a polymer loaded with an electrically conducting material. More preferably, the at least one plastics material element forms at least part (most preferably the whole) of, or is integral with, the amplification reaction vessel.

The method and apparatus of the invention are extremely versatile in their applications. They can be used to generate both quantitative and qualitative data regarding the target nucleic acid sequence in the sample, as discussed hereinbefore. In particular, not only does the invention provide for quantitative amplification, but it can also be used, additionally or alternatively, to obtain characterising data such as duplex destabilisation temperatures or melting points.

In a strand specific assay in accordance with the invention, the amplification reaction is conducted in the presence of an oligonucleotide probe which is specific for a region of the target sequence and which includes an electrochemical label, so as to allow the binding of the probe to the target sequence to be monitored. The expression “electrochemical label” used herein refers to any species or chemical moiety which produces a different electrochemical motif to that of native DNA.

Examples of electrochemical labels include modified base residues which have distinguishable electrochemical properties to native bases in the target sequence and otherwise present in the amplification reaction. Examples of such bases include inosine, xanthosine, hypoxanthine, xanthine, 1-methyladenine, 6-methyladenine, 6-benzyladenine, 8-oxyadenine, 2-aminopurine as well as bases which are otherwise modified for example by osmium tetroxide, pyridine or chloroacetaldehyde.

Alternatively, the probe may comprise a pair of labels which will undergo a detectable redox reaction when in close probity to each other. The probe may be in the form of a molecular beacon as described above or a variant of this such as a “Scorpion™” type probe where the molecular beacon is contiguous with an amplification primer. The probe may be a hydrolysis probe which is broken down during the amplification reaction in a manner similar to that of the TAQMAN™ probes. The probe may also comprise an oligonucleotide which comprises a first and second label and a site for a restriction enzyme which cuts at a specific double stranded DNA sequence located intermediate said first and second labels. Such probes can be utilised in conjunction, with a restriction enzyme using methods analogous to those described in WO-99/28501.

In yet a further alternative, the probe is a two-part probe (hybe-probe) also as described above, the two parts of which hybridise to the target sequence in close proximity to each other so that the labels are brought into proximity at that time.

In all of these cases, hybrisation of the probe to the target sequence will result in a change in redox properties as the relative spacing of the pair of labels changes. This change can be recorded by taking the electrochemical measurements in the course of the reaction.

In the method of the invention, the sample may be subjected to conditions under which the probe hybridises to the samples during or after the amplification reaction has been completed. The process allows the detection to be effected in a homogenous manner, in that the amplification and monitoring can be carried out in a single container with all reagents added initially. No subsequent reagent addition steps are required. Neither is there any need to effect the method in the presence of solid supports (although this is an option as discussed further hereinafter).

For example, where the probe is present throughout the amplification reaction, the electrochemical signal may allow the progress of the amplification reaction to be monitored. This may provide a means for quantitating the amount of target sequence present in the sample.

During each cycle of the amplification reaction, amplicon strands containing the target sequence bind to probe and thereby generate a signal. As the amount of amplicon in the sample increases, so the signal will increase. By plotting the rate of increase over cycles, the start point of the increase can be determined.

The probe may comprise a nucleic acid molecule such as DNA or RNA, which will hybridise to the target nucleic acid sequence when the latter is in single stranded form. In this instance, the amplification reaction conditions will include those which render the target nucleic acid single stranded. Alternatively, the probe may comprise a molecule such as a peptide nucleic acid or other nucleic acid analogue which is able to specifically bind the target sequence in double stranded form.

In particular, the amplification reaction used may involve a step of subjecting the sample to conditions under which any of the target nucleic acid sequence present in the sample becomes single stranded, such as PCR or LCR.

It is possible then for the probe to hybridise during the course of the amplification reaction, provided appropriate hybridization conditions (e.g. thermal or electrochemical conditions) are encountered. Examples of electrochemical conditions which may be employed to effect am amplification reaction are described in PCT/GB91/01563.

In a preferred embodiment, the probe may be designed such that these conditions are met during each cycle of the amplification reaction. Thus at some point during each cycle of the amplification reaction, the probe will hybridise to the target sequence and generate a signal. As the amplification proceeds, the probe will be separated or melted from the target sequence and so the signal generated will change. Thus the intensity of a signal will increase as the amplification proceeds because more target sequence becomes available for binding to the probe.

By monitoring the electrochemical signal during each cycle, the progress of the amplification reaction can be monitored in various ways. For example, the data provided by melting peaks can be analysed, for example by calculating the area under the melting peaks, and this data plotted against the number of cycles.

Depending on the nature of the assay being studied and the detection means being employed, the probe may either be free in solution or immobilised on a support, for instance on an electrode.

As discussed above, the probe may be designed such that it is hydrolysed by the DNA polymerase used in the amplification reaction thereby releasing the electrochemical label. This provides a cumulative signal, with the amount of free label present in the system increasing with each cycle.

In order to achieve a fully reversible signal which is directly related to the amount of amplification product present at each stage of the reaction, and/or where speed of reaction is of the greatest importance, for example in rapid PCR, it is preferable that the probe is designed such that it is released intact from the target sequence and so may take part again in the reaction. This may be, for example, during the extension phase of the amplification reaction. However, since the signal is not dependent upon probe hydrolysis, the probe may be designed to hybridise and melt from the target sequence at any stage during the amplification cycle, including the annealing or melt phase of the reaction. Such probes will ensure that interference with the amplification reaction is minimised.

Where probes which bind during the extension phase are used, their release intact may be achieved by using a 5′-3′ exonuclease lacking enzyme such as Stoffle fragment of Taq or Pwo. This may be useful when rapid PCR is required as hydrolysis steps are avoided.

When used in this way, it is important to ensure that the probe is not extended during the extension phase of the reaction. Therefore, the 3′ end of the probe is blocked, suitably by phosphorylation.

In a particular embodiment, the method of the invention is effected in the presence of a DNA binding agent which facilitates electrochemical measurements. In other words, the presence of these agents in the reaction mixture facilitates or creates sufficient differential in electrochemical properties as a result of amplification, that amplification can be monitored readily.

Examples of such agents include intercalating dyes such as ethidium bromide, SybrGold, SybrGreen, PiCoGreen or acridine orange, a single stranded binding protein such as E. coli SSB or cisplatin or an electrochemical dye such as Hoechst 33258.

In some instances, the electrochemical measurement may be the result of interaction between an added labelled or modified nucleotide nucleic acid probe and an amplicon incorporated labelled nucleotide or DNA binding agent present in the reaction.

The data generated in this way can be interpreted in various ways. In its simplest form, an increase in electrochemical signal from the amplicon in the course of or at the end of the amplification reaction is indicative of an increase in the amount of the target sequence present, suggestive of the fact that the amplification reaction has proceeded and therefore that the target sequence was in fact present in the sample. However, as outlined above, quantitation is also possible by monitoring the amplification reaction throughout. Finally, it is possible to obtain characterisation data and in particular melting point analysis, either as an end point measure or throughout, in order to obtain information about the sequence as will be discussed further below.

In a fourth aspect of the invention, there is provided a method for determining a characteristic of a target nucleic acid sequence, the method comprising carrying out a reaction as described above, and determining a particular reaction condition, characteristic of said sequence, at which the electrochemical measurement changes as a result of destabilisation or formation of a duplex in the amplification reaction.

Examples of characteristics which may be determined in this way are polymorphisms and/or allelic variations.

In a particular embodiment of the invention the probe may be used to quantitate RNA transcripts, for example in expression experiments, that may be used in drug discovery. In particular this embodiment is suitable for expression studies in tissues from eukaryotic organism. DNA encoding proteins in eukaryotic cells may contain introns, non-coding regions of DNA sequence, and exons that encode for protein sequence. Non-coding intron sequences are removed from RNA sequences that are derived from the DNA sequences during cellular “splicing” processes. PCR primers are normally targeted at coding regions and when reverse transcriptase PCR is used on total nucleic acid extracts, products will result from both DNA dependent amplification and RNA dependent amplification. Thus PCR alone, when used for expression studies, will contain amplification products resulting from genomic DNA and expressed RNA.

A probe that is designed to bind across introns, on adjacent terminal regions of coding exons, will have limited interaction because of the intron region. Spliced RNA has these regions removed and therefore the adjacent terminal regions of coding exons form one continuous sequence allowing efficient binding of the probe.

Conversely, a probe may detect only an amplification product of genomic DNA if it is designed such that it binds an intron region. Signal generated from such a probe would relate only to the DNA concentration and not the RNA concentration of the sample.

Thus in a further embodiment, the probe is specific either for a splice region of RNA or an intron in DNA, so that only one of amplified RNA or amplified DNA is detected and/or quantitated.

Suitable reaction conditions include temperature, or the response to the presence of particular enzymes or chemicals. By monitoring changes in electrochemical signals as these properties are varied, information characteristic of the precise nature of the sequence can be achieved. For example, in the case of temperature, the temperature at which the probe separates from the sequences in the sample as a result of heating can be determined. This can be extremely useful for example to detect, and if desired also to quantitate, polymorphisms and/or allelic variation in genetic diagnosis. “Polymorphisms” is intended to include transitions, transversions, insertions and deletions of inversions which may occur in sequences, particularly in nature.

The hysteresis of melting and hybridisation will be different if the target sequence varies by only one base pair. Thus for example, where a sample contains only a single allelic variant, the temperature of melting of the probe will be a particular value which will be different from that found in a sample which contains only another allelic variant. A sample containing both allelic variants which show two melting points corresponding to each of the allelic variants. Similar considerations apply with respect to electrochemical properties, or in the presence of certain enzymes or chemicals. The probe may be immobilised on a solid surface across which an electrochemical potential may be applied. Target sequence will bind to or be released from the probe at particular temperature values depending upon the precise nature of the sequence.

In addition, the kinetics of probe hybridisation will allow the determination, in absolute terms, of the target sequence concentration. Changes in electrochemical signal from the sample can allow the rate of hybridisation of the probe to the sample to be calculated. An increase in the rate of hybridisation will relate to the amount of target sequence present in the sample. As the concentration of the target sequence increases as the amplification reaction proceeds, hybridisation of the probe will occur more rapidly. Thus this parameter also can be used as a basis for quantification. This mode of data processing is useful in that it is not reliant on signal intensity to provide the information.

A fifth aspect of the present invention provides a probe for use in a method as described above.

A sixth aspect provides a kit for use in a method according to the first aspect, the kit comprising at least one reagent required for the amplification reaction, and either a probe according to the fifth aspect of the invention or a DNA binding agent as described above.

Such a kit may also comprise an amplification reaction vessel according to the third aspect of the invention, and/or apparatus according to the second.

The invention will now be particularly described by way of example with reference to the accompanying diagrammatic drawings in which:

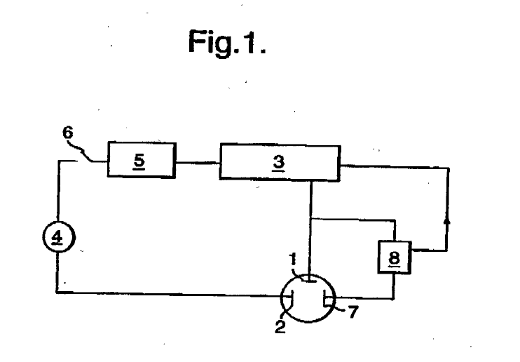

FIG. 1 is a circuit diagram of a circuit suitable for taking electrochemical measurements in the context of the method of the invention;

FIG. 2 is a diagrammatic section of a thermally controlled electrochemical cell for use in the method of the invention;

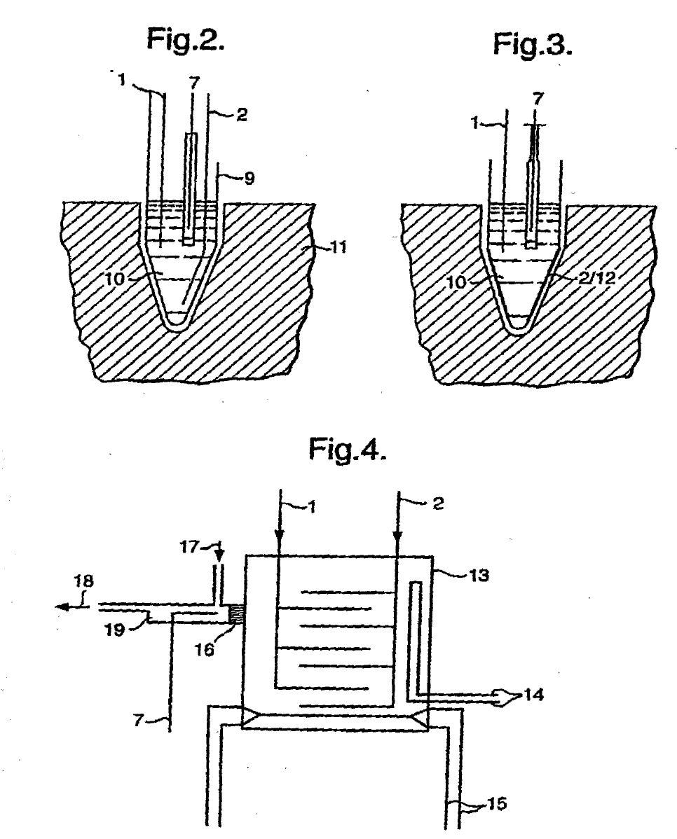

FIG. 3 is a diagrammatic section of an alternative thermally controlled electrochemical cell for use in the method of the invention; and

FIG. 4 is a diagrammatic section of a further alternative thermally controlled electrochemical cell for use in the method of the invention.

In the circuit shown in FIG. 1, a working electrode (1) is connected to a secondary electrode (2) by way of a potentiostat (3) and an ammeter (4). The circuit is connected to a direct current supply (5) and includes a switch (6). A reference electrode (7, such as a silver/silver chloride electrode or a calomel electrode, is connected in parallel as illustrated by way of a high impedance voltammeter (8).

In this arrangement, the potential of the working electrode and the current can be measured by reading the voltammeter (8). The working electrode potential within the cell is controllable by means of the potentiostat (3).

FIG. 2 illustrates one form of the electrochemical cell in more detail. In this instance the working electrode (1), the secondary electrode (2) and the reference electrode (7) project into a vessel (9) which contains the amplification reaction mixture (10). The vessel (9) is fitted into a suitable aperture in a heating block (11). In this way, the reaction mixture (10) may be thermally cycled in an amplification reaction in the usual way. The effects of this process of the electrochemical characteristics of the mixture can be monitored to provide information about the progress of the reaction.

The embodiment of FIG. 3 differs in that in this instance, at least a part (12) of the reaction vessel comprises an electrically conducting polymer. By passing a current through the polymer, controlled heating of the reaction mixture (10) is effected. In addition, the polymer acts as the secondary electrode (2).

Finally, in the embodiment of FIG. 4, a microfabricated reaction vessel (13) produced by lithographic etching of silicon wafers is provided. Working electrode (1) and secondary electrode (2) and reference electrodes (7) are deposited on the vessel (13). An integral heater element (14) and resistive thermal sensor (15) provide means for thermal control of the reaction vessel (13). An etched frit (16) and inlet (17) and outlet (18) provide a means for replenishing and maintaining reference electrolyte (19).

The frit (16) provides a salt bridge facilitating a fixed potential reference electrode arrangement.

1. A method for detecting a target nucleic acid sequence in a sample, the method comprising subjecting the sample to an amplification reaction and taking continuous electrochemical measurements on the sample in situ during the amplification reaction, wherein the amplification reaction is conducted in the presence of an oligonucleotide probe that is specific for a region of the target nucleic acid sequence and includes an electrochemical label, which allows binding of the probe to the target sequence to be electrochemically monitored wherein a change in electrochemical measurement indicates the presence of the target in the sample.

2. The method according to claim 1, wherein the electrochemical label comprises a modified base residue which has different electrochemical properties to native bases in the target sequence and otherwise present in the amplification reaction.

3. The method according to claim 2, wherein the modified base residue is inosine, xanthosine, hypoxanthine, xanthine, 1-methyladenine, 6-methyladenine, 6-benzyladenine, 8-oxyadenine or 2-aminopurine or a base which is modified by osmium tetroxide, pyridine or chloroacetaldehyde.

4. The method according to claim 1, wherein the probe comprises a pair of labels which will undergo a detectable redox reaction when in close proximity to each other.

5. The method according to claim 4, wherein the probe comprises an oligonucleotide having complementary 5′ and 3′ sequences optionally contiguous with an amplification primer, a probe which is hydrolysed during the amplification reaction, a two-part probe the parts of which hybridise in close proximity to each other on the target sequence, or a probe having first and second labels separated by a region which forms a site for a restriction enzyme when in double stranded form.

6. The method according to claim 1, wherein the probe is designed such that it can be released intact from the target sequence and so may take part again in the reaction.

7. The method according to claim 1, wherein the probe is free in solution.

8. The method according to claim 1, wherein the probe is specific either for a splice region of RNA or an intron in DNA, so that only one of the amplified RNA or amplified DNA is detected.

9. The method according to claim 1, wherein the amplification reaction is effected in the presence of a DNA binding agent which facilitates electrochemical measurement.

10. The method according to claim 9, wherein the DNA binding agent comprises an intercalating dye such as ethidium bromide, SybrGold, SybrGreen, PicoGreen, or acridine orange, a single stranded binding protein such as E. coli SSB, cisplatin or an electrochemical dye such as Hoechst 33258.

11. The method according to claim 9, wherein the electrochemical measurement is a result of interaction between the probe and the DNA binding agent.

12. The method according to claim 1, further comprising determining a particular reaction condition, characteristic of said sequence, at which the electrochemical measurements change as a result of destabilization or formation of a duplex in the amplification reaction wherein the change in electrochemical measurement is determinative of the characteristic of the target sequence.

13. (canceled)

14. The method according to claim 1, wherein the target sequence is an internal control sequence.

15. The method according to claim 1, wherein the electrochemical measurements are temperature dependent electrochemical measurements.

16. The method according to claim 1, wherein the electrochemical measurements are potentiometric, conductometric or amperometric, coulometric, and/or voltametric or polarographic measurements.

17. The method according to claim 1, wherein the electrochemical measurements are used to determine whether and/or to what extent an amplification reaction has taken place.

18. The method according to claim 1, wherein the electrochemical measurements are used to quantitate the amount of the target nucleic acid sequence in the sample.

19. The method according to claim 1, wherein the electrochemical measurements are temperature dependent electrochemical measurements which allow characterization of amplification species.

20-22. (canceled)

23. A probe for use in a method for detecting a target nucleic acid sequence in a sample, wherein the probe is specific for a region of the target nucleic acid sequence and includes an electrochemical label, which allows binding of the probe to the target sequence to be electrochemically monitored, and the probe is free in solution.

24. An apparatus for use in a method for detecting a target nucleic acid sequence in a sample, the apparatus comprising (i) an amplification reaction vessel which comprises an electrochemical cell, (ii) means for taking continuous electrochemical measurements on a sample contained in the amplification reaction vessel and (iii) temperature control and measurement means, wherein the electrochemical cell comprises at least one element formed from an electrically conducting plastics material.

25-45. (canceled)
